(19) United States
(12) Patent Application Publication
(10) Pub. No.: US 2021/0121681 A1
(43) Pub. Date: Apr. 29, 2021

(54) INCREASED MILK PRODUCTION

(71) Applicant: MilkStim, Inc., Napa, CA (US)

(72) Inventors: Howard J. Leonhardt, Corona Del Mar, CA (US); Paul Norman, Napa, CA (US); Jorge Genovese, Buenos Aires (AR); Leslie Miller, Tampa, FL (US)

(21) Appl. No.: 17/044,013

(22) PCT Filed: Apr. 1, 2019
(86) PCT No.: PCT/US2019/025177

§ 371 Date: Sep. 30, 2020

Related U.S. Application Data
(60) Provisional application No. 62/650,948, filed on Mar. 30, 2018.

(51) Int. Cl. A61N 1/04; A61N 1/32; A61N 1/36; A61M 5/142

(52) U.S. Cl. CPC A61N 1/0464; A61N 1/326; A61M 2210/1007; A61M 5/14248; A61N 1/36034

(57) ABSTRACT

A method of using a bioelectric stimulator for delivering an electrical signal to a subject's tissue, wherein the bioelectric stimulator utilizes the electrical signal to precisely control protein expression and/or release in the tissue on demand so as to increase milk production in a subject, the method including: delivering selected electrical signals to the subject so as to precisely control protein expressions and/or release to increase milk production in the subject.

### CROSS-REFERENCE TO RELATED APPLICATIONS

This application is a national phase entry under 35 U.S.C. § 371 of International Patent Application PCT/US2019/025177, filed Apr. 1, 2019, designating the United States of America and published as International Patent Publication WO 2019/191756 A1 on Oct. 3, 2019, which claims the benefit of the filing date of U.S. Provisional Patent Application 62/650,948, filed on Mar. 30, 2018, the contents of which are incorporated herein by this reference.

### TECHNICAL FIELD

The disclosure relates generally to the field of medical devices and associated treatments, systems, and methods, and more specifically to precise bioelectrical stimulation of a subject's mammary tissue to enhance milk production (lactation). This bioelectric stimulation may be optionally augmented with the administration of a composition comprising, among other things, stem cells and nutrients, useful to stimulate and treat the subject, the subject's tissue(s), the subject's organ(s), and/or the subject's cells to, e.g., enhance milk production/lactation.

### BACKGROUND

The mammary glands of a mammal produce milk for feeding the young. Oftentimes, natural milk production can be insufficient however. It would be an improvement in the art to be able to increase milk production on an individual basis.

### BRIEF SUMMARY

Described herein is a method of increasing milk production in a suitable female subject, the method comprising: delivering a selected electrical signal to the subject so as to increase milk production in the subject. In such a method, the selected electrical signal is typically 250 μA, 100 Hz, bipolar, applied for 60 minutes every other day for a month. Alternatively, the selected electrical signal may be 250 μA, 100 Hz, bipolar, applied for 30 minutes every other day for a month.

Further described is a bioelectric stimulator programmed to activate expression and/or release in a subject of, e.g., SDF-1, IGF-1, and VEGF. Application of such a bioelectric stimulator to or about the mammary glands or breasts of a female mammal (e.g., a human or other mammal such as a sheep, cow, or dog) leads to increased milk production by the female.

Described is a bioelectric stimulator including: a power source (e.g., battery, capacitor, or other suitable source of electricity), and means for delivering an electrical signal to a subject's tissue (e.g., via electrode(s) or wirelessly). The bioelectric stimulator utilizes the electrical signal to precisely control particular protein expression or release in the tissue on demand. Such a bioelectric stimulator preferably precisely controls release of protein in the subject, without a diminishing effect over time.

Also described is a method of using the bioelectric stimulator to stimulate milk production in a subject, the method including: delivering selected electrical signals to or near the subject's mammary gland(s), teats, and/or breast area so as to precisely control protein expressions in the right sequence and volume. Such a method can further include separately delivering to the subject a cocktail of regenerative agents including any combination of the following: stem cells, endothelial progenitor cells, selected exosomes, selected alkaloids, selected anti-inflammatory agents, nutrient hydrogel, organ specific matrix, selected growth factors, amniotic fluid, placenta fluid, cord blood, and embryonic sourced growth factors and cells.

Also described is a method of using the bioelectric stimulator to achieve a desired result in a subject, wherein the desired result is selected from the group consisting of milk production, breast tissue generation or regeneration, and any combination thereof.

Particularly described is a system that includes:
a. A bioelectric stimulator that controls/stimulates, e.g., the release/production of SDF-1, IGF-1, and VEGF.
b. A micro infusion or other pump (e.g., a FluidSync™ micropump available from Fluidsynchrony of Pasadena, Calif., US), which is programmable and re-fillable and preferably has a low cell damage design. Such a pump preferably includes a refilling silicon septum port or ports and reservoir chambers.
c. A multi-component composition that includes adipose-derived stem cells, muscle-derived stem cells (when needed for muscle), exosomes, Micro RNAs, nutrient hydrogel, growth factor cocktail, organ specific matrix, selected alkaloids, and/or selected anti-inflammatory agents.

The pump and stimulator may be associated with (e.g., connected to) the organ to be treated/regenerated with a pacing infusion lead (e.g., one available from Nanoscribe of Eggenstein-Leopoldshafen, Germany). A conductive soft wrap can be used for certain applications herein.

The stimulator can be designed to externally deliver all protein enhancing signals wirelessly to the subject's organ(s), tissue(s), and/or cells.

In certain embodiments, such a device may utilize bioelectric signals delivered wirelessly to the organ(s), tissue(s), and/or cell(s) being treated. Such a device may utilize bioelectric organ regeneration signals delivered via the nervous system of the subject being treated.

In certain embodiments, described is a bioelectric stimulator that is programmable to deliver specific electrical signals for use in increasing the production of milk in breast tissue.

As further described herein, after use of the bioelectric stimulator herein, the subjects exhibited an excellent health condition including with the mammary glands, even two months after treatment (clinical and echo control). The desired effects persisted without adverse undesired effects.

### BRIEF DESCRIPTION OF THE DRAWINGS

FIG. 1 depicts a programmed bioelectric stimulator for delivery of electrical stimulation to the subject (as per the Example).

FIG. 2 depicts a different programmed bioelectric stimulator depicted alongside a U.S. quarter.

FIG. 3 depicts a micropump for optional use with the system.

FIG. 4 depicts an interface for use with a system that incorporates a pump.

FIG. 5 depicts a combination bioelectric stimulation and stem cells and growth factors infusion catheter.

FIG. 6 is a close up view of a conductive and infusion cork screw tip, which may be used with the catheter system of FIG. 5.

FIG. 7 depicts an image of the signal (voltage and frequency) associated with the expression and/or release of VEGF: 100 mV, 50 Hz, square wave.

FIG. 8 depicts an image of the signal (voltage and frequency) associated with the expression and/or release of SDF-1 (2^{nd} part): 0.25 mA (3.0V shown here), 100 Hz, 100 μs pulse width, square wave.

FIG. 9 depicts an image of the signal (voltage and frequency) associated with the expression and/or release of IGF-1: 3.0 mV, 22 Hz, square wave.

FIG. 10 depicts an image of the signal (voltage and frequency) associated with the expression and/or release of SDF-1: 3.5 mV, 30 Hz, square wave.

FIG. 11, Panels A-C depict the histology results of Example I, where CD34 cells are depicted Hematoxylin-eosin (“H-E”). 4λ. FIG. 11, Panel A, Control. FIG. 11, Panel B, Electrical Stimulation. FIG. 11, Panel C, Control in the animal treated just in one gland.

FIG. 12, Panels D-G depicts the histology results of Example I, where CD34 cells are depicted. FIG. 12, Panels D and E, Control. FIG. 12, Panels F and G, Electrical Stimulation.

FIG. 13 depicts the raw data of milk production for the three cows of Example II in tabular form.

FIG. 14 displays the results of Example II graphically.

FIG. 15 shows the increase in milk production of the three cows of Example II in a bar graph.

### DETAILED DESCRIPTION

In a preferred embodiment, the device measures and records the electrical activity that stimulates milk production after suckling action on a breast nipple. The device then “plays” this same series of bioelectric signals (“electrical activity”) back to the subject for an extended period of time and the signal may be amplified for greater potency/effect.

In a preferred embodiment, the device causes controlled (on demand) release/expression in the subject of natural milk production enhancing hormones.

In a preferred embodiment, a specialized bra (not shown) is placed onto the subject. Such a bra is otherwise typical, but further includes, e.g., an electro conductive gel layer that surrounds the exterior of the breast(s). The electro conductive gel layer is in electrical connection with the programmed bioelectric stimulator via, e.g., leads. Such a bra is used to deliver the desired bioelectrical signal(s) to the subject.

In a preferred embodiment, a “matrix” or composition for administration may be included that comprises adipose-derived stem cells, bone marrow-derived stem cells, muscle-derived stem cells (e.g., when needed for muscle), exosomes, MicroRNAs, nutrient hydrogel, growth factor cocktail, organ specific matrix, selected alkaloids, and/or selected anti-inflammatory agents.

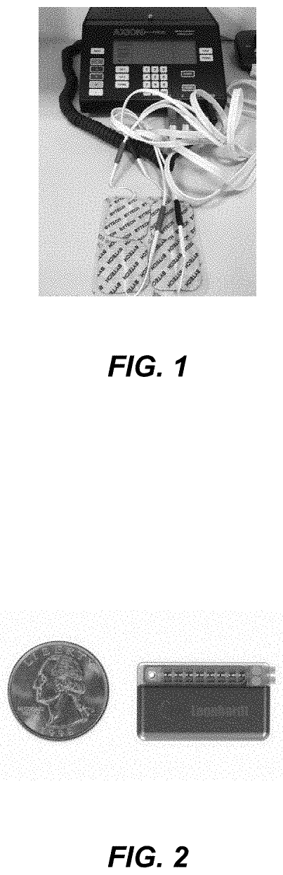

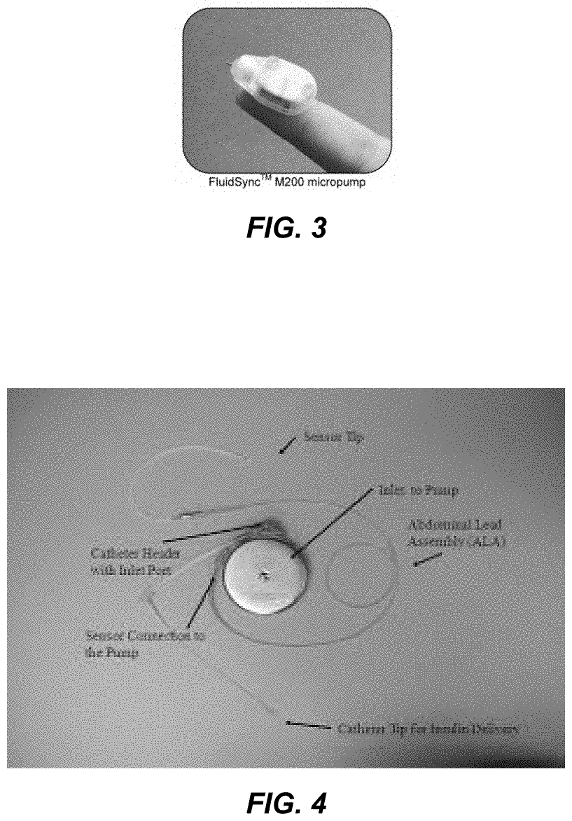

Referring now to FIG. 1, depicted is a stimulator, conductive electrode patch and connecting leads. Such a stimulator programmable to produce bioelectric signals is available from QIG Greatbatch/Greatbatch, Inc. of Frisco, Tex., US. Any associated microinfusion pump (e.g., FIG. 3) is preferably programmable and re-fillable with low cell damage design. Refilling may be by silicon septum ports and reservoir chambers. The microinfusion pump (e.g., FIG. 3) for continuous or repeat delivery of a liquid composition, which microinfusion pump may include silicon septum ports and associated reservoir chambers connected to the bioelectric stimulator microinfusion pump to the tissue with a pacing infusion lead.

A similar system is currently being investigated for various other applications. See, e.g., U.S. patent application Ser. No. 15/812,760, filed Nov. 14, 2017, which application is a continuation-in-part of U.S. patent application Ser. No. 15/460,129, filed on Mar. 15, 2017 (U.S. 2017/0266371A1, Sep. 21, 2017), the contents of the entirety of each of which are incorporated herein by this reference.

A “matrix”, as used herein, is a liquid composition for delivery by the micropump or pump, which matrix believed to aid in stem cell differentiation, but in any event may to be useful in the composition. See, e.g., Procházka et al. “Therapeutic Potential of Adipose-Derived Therapeutic Factor Concentrate for Treating Critical Limb Ischemia,” Cell Transplantation, 25(9):1623-1633(11) (2016) and “Cocktail of Factors from Fat-derived Stem Cells Shows Promise for Critical Limb Ischemia,” world wide web at sciencenewsline.com/news/2016012204520017.html (Jan. 22, 2016), the contents of each of which are incorporated herein by this reference. Repeated doses of the composition may be used.

Generally, the system hereof (for the breasts to increase milk production) involves a bioelectric stimulator controlling expression and/or release of, for example, SDF-1, IGF-1, VEGF, or any combination thereof by the subject's breast tissue (e.g., mammary gland). SDF-1 generally recruits stem cells and matures blood vessels. IGF-1 is for DNA repair. VEGF grows blood vessels. The protein expression may be used together.

The micro voltage signal generator may be produced utilizing the same techniques to produce a standard heart pacemaker well known to a person of ordinary skill in the art. An exemplary microvoltage generator is available (for experimental purposes from Cal-X Stars Business Accelerator, Inc. DBA Leonhardt's Launchpads or Leonhardt Vineyards LLC DBA Leonhardt Ventures of Salt Lake City, Utah, US). The primary difference is the special electrical stimulation signals needed to control, e.g., precise protein release on demand (which signals are described later herein). The leading pacemaker manufacturers are Medtronic, Boston Scientific Guidant, Abbott St. Jude, BioTronik and Sorin Biomedica.

Construction of the electric signal generators and pacemakers, are known in the art and can be obtained from OEM suppliers as well as their accompanying chargers and programmers. The electric signal generators are programmed to produce specific signals to lead to specific protein expressions at precisely the right time for, e.g., optimal organ treatment or regeneration.

An infusion and electrode wide area patch may be constructed by cutting conduction polymer to shape and forming plastic into a flat bag with outlet ports in strategic locations.

A wireless, single lumen infusion pacing lead (or leads) and/or infusion conduction wide array patch(es) may all be used to deliver the signals and substances to the subject or they may be used in combination.

A re-charging wand for use herein is preferably similar to the pacemaker re-charging wand developed by Alfred Mann in the early 1970's for recharging externally implantable pacemakers.

Micro infusion pumps (e.g., FIG. 3) can be purchased or produced in a manner similar to how they have been produced for drug, insulin, and pain medication delivery since the 1970's. The programming computer can be, e.g., a standard laptop computer. The programming wand customary to wireless programming wands may be used to program heart pacers.

As depicted in FIG. 4, an interface for use with a system that incorporates a micropump can include a sensor tip, abdominal lead assembly, catheter tip for infusion delivery to the subject tissue, sensor connection to the pump, and catheter header with inlet port.

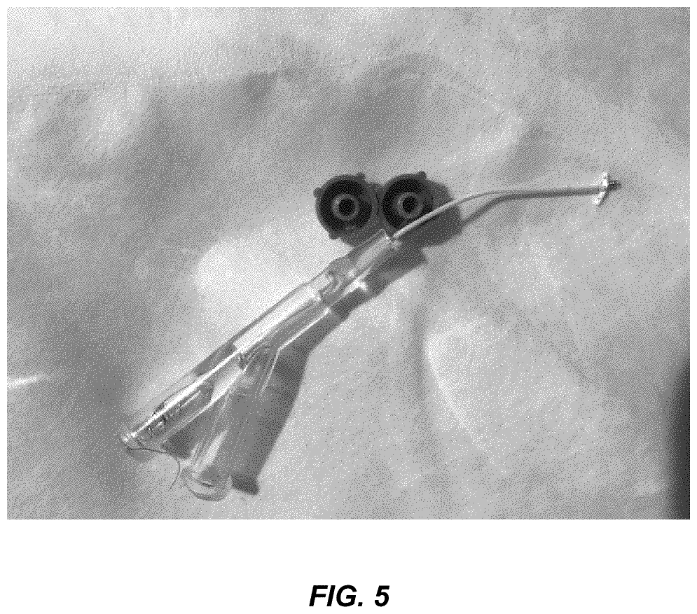

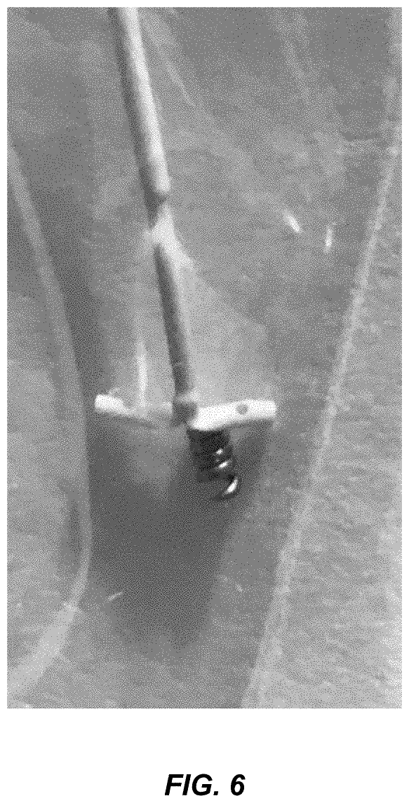

FIG. 5 depicts a combination bioelectric stimulation and stem cell and growth factor(s) infusion catheter usable with the described system. A corkscrew tip (see, e.g., FIG. 6) may be of a standard type utilized to secure most heart pacemakers in heart tissue. Wireless delivery of the signal or electro-acupuncture needle delivery is included. FIG. 6 is a close up of the conductive and infusion cork screw tip for getting deep into target tissue. The tip includes suture tabs for even more secure fixation to the target organ.

In use, the bioelectric stimulator is attached to the breast tissue, actuated, and runs through programmed signals to signal the release of, e.g., SDF-1 by the breast tissue such as the mammary gland.

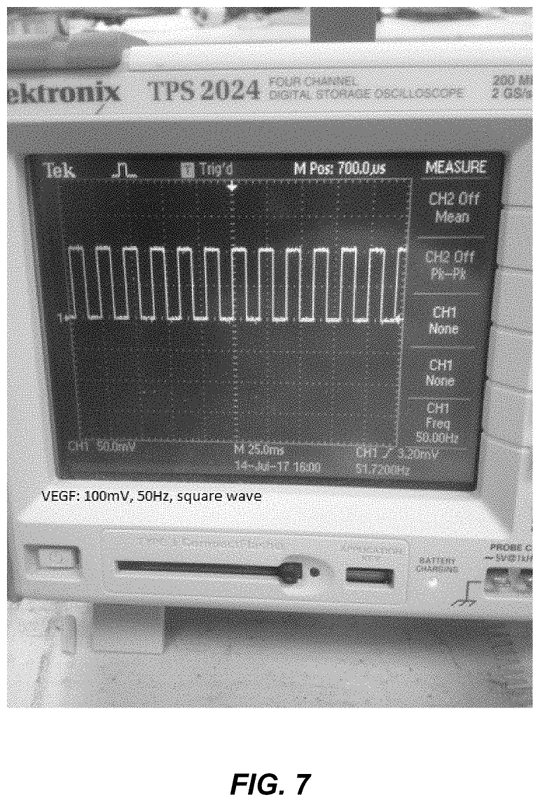

In such a method, when the electrical signal includes (within 15%): 0.1V applied at a frequency of about 50 Hz with a duration of about three (3) minutes (wherein the electrical signal is as measured three (3) mm deep into the tissue), the protein produced is VEGF (FIG. 7).

In such a method, when the electrical signal includes (within 2%): 200 picoamps for about 10 seconds for about one (1) hour and the pulse has an amplitude of about 5 volts and a width of about 0.5 milliseconds for about 1 hour, with a duration of about one (1) minute (wherein the electrical signal is as measured three (3) mm deep into the tissue), stem cells differentiate.

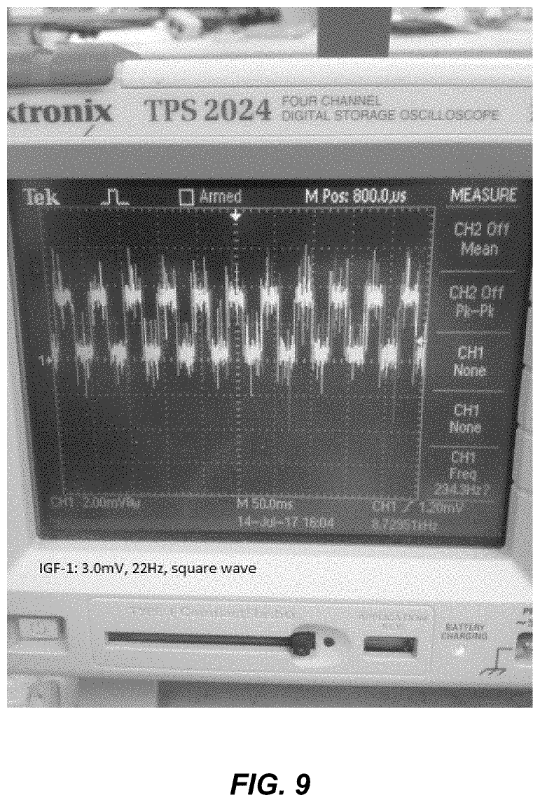

In such a method, when the electrical signal includes (within 15%): 3 mv with a frequency of about 22 Hz, and a current of about 1 mA for about fifteen (15) minutes and 3 ma for about fifteen (15) minutes (duration 5 minutes) (wherein the electrical signal is as measured three (3) mm deep into the tissue), the protein produced is IGF-1 (FIG. 9).

For example, upregulation of IGF-1, VEGF, and SDF-1 was achieved in cardiomyoctyes using such signals. Upregulation of SDF-1 was achieved in pig heart. Upregulation of VEGF was achieved in eye cells.

Also described is a method of activating a tissue to produce stromal cell-derived factor 1 (“SDF-1”), the method including: stimulating the (e.g., human breast) tissue with an electrical signal, wherein the electrical signal includes (within 15%): 30 pulses per second with a voltage of about 3.5 mV, and successively alternating currents of about 700 to 1500 picoamps for about one minute, and again with 700 to 1500 picoamps for about one minute and stimulated with current of about 0.25 mA, pulse duration of about 40 pulses/s, pulse width of about 100 μs, wherein the electrical signal is as measured three (3) mm deep into the tissue.

Further described is a method of activating a tissue to attract a stem cell, the method including: stimulating the (e.g., human) tissue with an electrical signal, wherein the electrical signal includes (within 2%): fifteen (15) mV and a current of about 500 picoamps at 70 pulses per minute for about three (3) hours and 20 pulses per minute, a pulse amplitude of from about 2.5-6 volts, and a pulse width of from about 0.2-0.7 milliseconds for about three (3) hours for about three (3) minutes, wherein the electrical signal is as measured three (3) mm deep into the tissue.

In some cases, SDF-1 recruits via a presumed homing signal new reparative stem cells to the tissue. VEGF causes new nutrient and oxygen producing blood vessels to grow into the tissue. IGF-1 repairs damaged cells, tissues, and organs. All of these proteins work together to fully regenerate an organ over time.

In such a method, the period of time is typically at least 24 hours.

In such a method, the field strength is typically at least 1 V/cm.

What follows are signals from the stimulator. For example, described are two PDGF expression control signals, one low voltage and one higher voltage. The test tissue is sheep heart tissue. The test cells are mesenchymal stem cells.

VEGF—Blood vessel sprouting growth: 0.1V applied at a frequency of 50 Hz. Duration 3 minutes.

SDF-1—Stem cell recruiting signal: 30 pulses per second with a voltage of 3.5 mV, and successively alternating currents of 700 to 1500 picoamps for one minute, and again with 700 to 1500 picoamps for one minute and stimulated with current of 0.25 mA, pulse duration of 40 pulses/s, pulse width of 100 μs, and frequency of 100 Hz—each signal for 40 minutes to 8 hours a day for 2 to 36 months as needed for ideal results. Duration 7 minutes.

Stem cell proliferation signals: 15 mV and a current of 500 picoamps at 70 pulses per minute for 3 hours and 20 pulses per minute, a pulse amplitude of from 2.5-6 volts, and a pulse width of from 0.2-0.7 milliseconds for 3 hours. Duration 3 minutes.

Stem cell differentiation signals to become muscle: 200 picoamps for 10 seconds for 1 hour and the pulse has an amplitude of 5 volts and a width of 0.5 milliseconds for 1 hour. Duration 1 minute.

Another method is to reverse polarity and drop the voltage.

IGF-1: 3mv with electric frequency of 22 Hz, and electric current of 1 mA for 15 minutes and 3ma for 15 minutes. Duration 5 minutes.

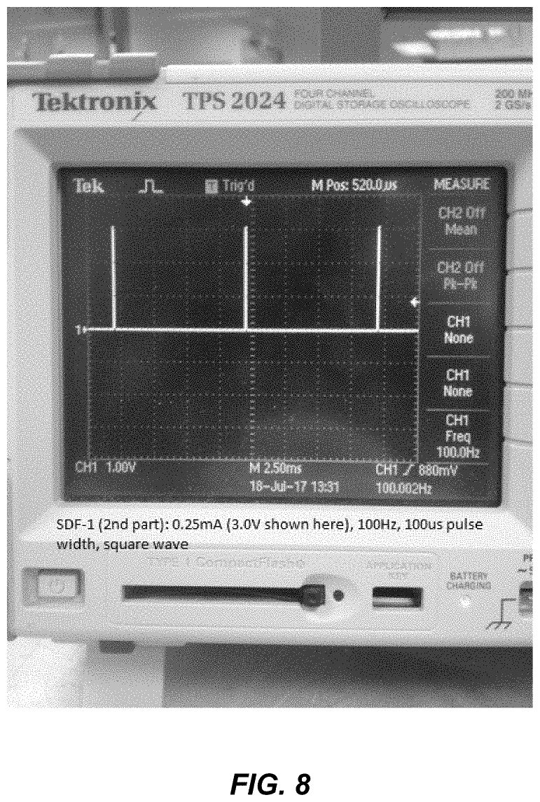

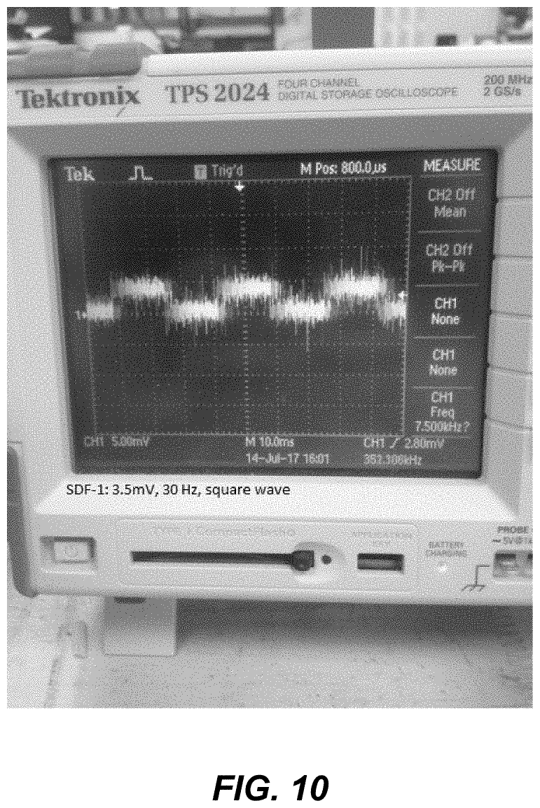

Specifically, FIG. 9 depicts an image of the signal (voltage and frequency) associated with the expression and/or release of IGF-1: 3.0 mV, 22 Hz, square wave. FIG. 10 depicts an image of the signal (voltage and frequency) associated with the expression and/or release of SDF-1: 3.5 mV, 30 Hz, square wave. FIG. 7 depicts an image of the signal (voltage and frequency) associated with the expression and/or release of VEGF: 100 mV, 50 Hz, square wave. FIG. 8 depicts an image of the signal (voltage and frequency) associated with the expression and/or release of SDF-1 (2^{nd} part): 0.25 mA (3.0V shown here), 100 Hz, 100 μs pulse width, square wave.

In certain embodiments, a subject's organ(s) and/or tissue(s) (e.g., the breast area) are first scanned or analyzed with a device to determine what her needs may be before treatment begins. The scanning/analysis can be by, e.g., measuring transmembrane voltage potential of a cell (see, e.g., Chernet & Levin, “Transmembrane voltage potential is an essential cellular parameter for the detection and control of tumor development in a Xenopus model,” Dis. Models & Mech. 6, pp. 595-607 (2013); doi:10.1242/dmm.010835, the contents of which are incorporated herein by this reference. See, also, Brooks et al. “Bioelectric impedance predicts total body water, blood pressure, and heart rate during hemodialysis in children and adolescents,” J. Ren. Nutr. 18(3):304-311 (May 2008); doi: 10.1053/j.jm.2007.11.008, the contents of which are incorporated herein by this reference, describing the use of bioelectric impedance to evaluate the variability of blood pressure, systolic blood pressure, etc.

In one embodiment, for instance, a calf sucks on the subject's breast and then the electrical activity going to and from the brain in the entire region of the mammillary network is measured, which signals will then be played back to the subject to stimulate milk production. See, e.g., Harun Yahya-Adnan Oktar, The Miracle of Hormones, page 44 (A9 Group, 2010).

Alternatively, plasma prolactin may be measured in the mammal after electrical stimulation. An increase in the levels of prolactin provides evidence of an increase in the stimulus of milk production. Such a procedure may be combined, e.g., with ultrasound/echo evaluation of, e.g., a cow utter.

The disclosure is further explained by the following illustrative Examples.

### Example I

Ovine Breast Electrical Stimulation (a Pilot Study on Large Animals with Inguinal Bilateral Breasts)

The objectives of the study were to evaluate the effect of electrical stimulation (“ES”) on potential breast tissue growth, to determine the tolerance to electrical stimulation on ovine breast, and to evaluate possible histological changes in mammary tissue with special attention on angiogenesis modulation and effects on stem cells populations.

Material and Methods: Animals. Three nulliparous female sheep, Romney marsh, in reproductive age (human equivalent around 22 years) with an average body weight of 85 pounds (38.55 kg).

A trained veterinarian evaluated treatment tolerance. This professional was in charge of external mammary glands evaluation. A veterinarian having experience with ultrasound of livestock was in charge of initial and final echo evaluation. A third veterinarian collected the biopsy specimens. The last two veterinarians and the pathologist were “blinded” as to the role of sheep in the experiment.

Dorsoventral, Lateromedial, and volume were evaluated by ultrasound at day 0 and day 30.

Self-adhering electrodes (FIG. 1) from a bioelectrical stimulator (e.g., a Ventura stimulator) were applied on each side of the mammary gland. Every other day for a month, the following stimulus was applied for 60 minutes: 250 μA, 100 Hz, bipolar.

The animals were treated as follows:

Sheep #1: Control (the electrodes were attached, but not connected to the bioelectric stimulator).

Sheep #2: Both breasts were treated with the bioelectric stimulator.

Sheep #3: Right breast treated, left breast not treated with the bioelectric stimulator.

The biopsy samples were fixed and evaluated using hematoxilin-eosin and the following antibodies: GOAT Anti-mouse IgG H8L (FITC) pre-absorber AB7064, Anti-CD105 Antibody (8A1) AB156756, and anti-CD34 Antibody (EP373Y) AB81289.

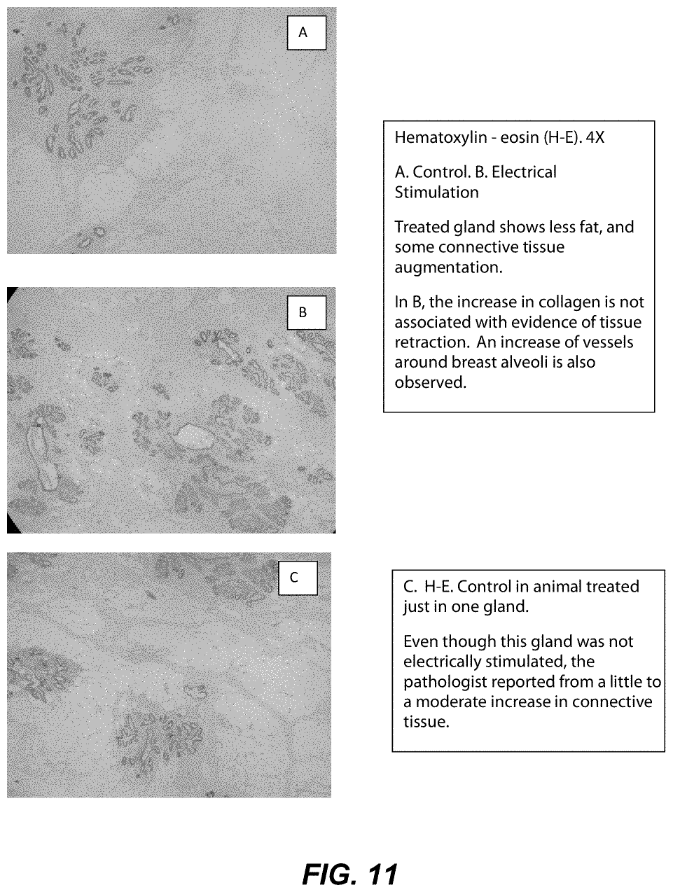

Results: Histology results are depicted in FIG. 11, Panels A-C, Hematoxylin-eosin (“H-E”). 4× FIG. 11, Panel A, Control. FIG. 11, Panel B, Electrical Stimulation. The treated gland shows less fat, and some connective tissue augmentation. In FIG. 11, Panel B, the increase in collagen is not associated with evidence of tissue retraction. An increase of vessels around breast alveoli is also observed. In FIG. 11, Panel C. Hematoxylin-eosin. Control in animal treated just in one gland. Even though this gland was not electrically stimulated, the pathologist reported a little to a moderate increase in connective tissue.

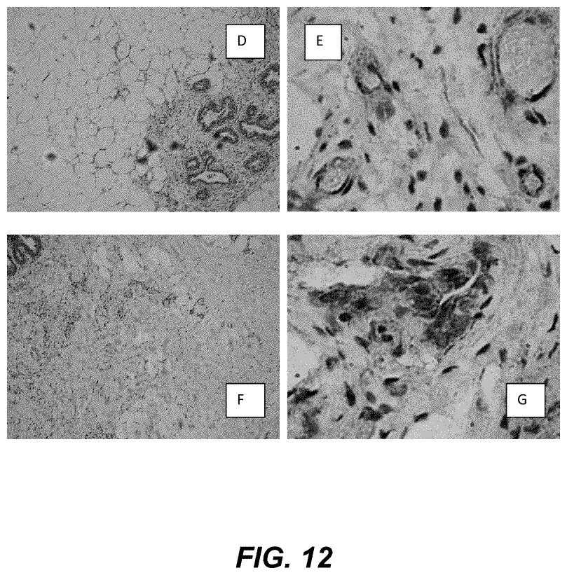

CD34 cells. FIG. 12, Panels D and E, Control. FIG. 12, Panels F and G, Electrical Stimulation. At high magnification, the presence of groups of positive cells in the tissue from the treated animal is evident. In the control, most of the positive cells were found in association with vessels.

CD105 cells. The pathologist reported that her evaluation confirmed the presence of CD105 positive cells in samples from treated animals. With the above mentioned limitation, no CD105 cells were found in the control samples.

Conclusions: This was a pilot trial to evaluate the effects of electrical stimulation on sheep's mammary glands. The animals did not show any sign of pain or discomfort during the treatment. No other adverse reaction, local or systemic, was detected.

The reduction of mammary gland size in some animals was interpreted by the veterinarians present to be due to an effect of changes in the photoperiod of the sheep while the experiment was being conducted. Photoperiod is critical in the ovine, and determines changes in their sexual organs. This aspect is especially important in the evaluation of the results of the animal treated in only one gland (Sheep #3). The increase in the treated side is apparently minor, but this animal appears to be in a clearly regressive mammary period in view of the changes in the untreated side.

The histology showed an increase of connective tissue without retracted areas. Even more, an increase in the laxity due to a reduction in the stromal cells population was observed. At the parenchyma, an increase of ductus and vessels was evident. Despite some limitations due to the restricted access to specific reactives, CD34+ cells that in the control were around the vessels in limited numbers were found in different areas of the treated tissue. Isolated CD105+ were observed in the parenchyma of treated glands. Neither signs of inflammation nor other pathological conditions were reported.

Results

Sheep #1: Control

| | Right | | |
|---|---|---|---|
| | Day | Day | Variation |
| | 0 | 30 | (%) |
| Dorsoventral (mm) | 16.1 | 17.7 | 9.94 |
| Lateromedial (mm) | 41 | 42.3 | 3.17 |
| Area (mm2) | 655 | 697 | 6.42 |

| | Left | | |
|---|---|---|---|
| | Day | Day | Variation |
| | 0 | 30 | (%) |
| Dorsoventral (mm) | 16.16 | 17.7 | 9.53 |
| Lateromedial (mm) | 35 | 30 | −14.3 |
| Area (mm2) | 478 | 451 | −6.35 |

Sheep #2: Both breasts treated

| | Right | | |
|---|---|---|---|
| | Day | Day | Variation |
| | 0 | 30 | (%) |
| Dorsoventral (mm) | 36.9 | 43.3 | 17.34 |
| Lateromedial (mm) | 47.5 | 47.5 | 0 |
| Area (mm2) | 715 | 869 | 21.54 |

| | Left | | |
|---|---|---|---|
| | Day | Day | Variation |
| | 0 | 30 | (%) |
| Dorsoventral (mm) | 20.2 | 28.1 | 39.1 |
| Lateromedial (mm) | 39.5 | 45.3 | 14.68 |
| Area (mm2) | 560 | 730 | 30.4 |

Sheep #3: Right breast treated, left breast not treated.

| | Right | | |
|---|---|---|---|
| | Day | Day | Variation |
| | 0 | 30 | (%) |
| Dorsoventral (mm) | 36.9 | 37.9 | 2.71 |
| Lateromedial (mm) | 34.6 | 44.8 | 29.47 |
| Area (mm2) | 720 | 764 | 5.5 |

| | Left | | |
|---|---|---|---|
| | Day | Day | Variation |
| | 0 | 30 | (%) |
| Dorsoventral (mm) | 24.7 | 27.1 | 9.72 |
| Lateromedial (mm) | 44.2 | 37.9 | −5% |
| Area (mm2) | 715 | 559 | −21.81% |

### Example II

This Example shows the effect of electrical stimulation on milk production in three treated cows.

Each of three cows underwent electrical stimulation for a two week period. An electrical stimulator was connected to the cows as described herein. Due to the volume and to maintain physiological lactation, stimulation was confined to one of four udders. Every other day for two weeks, the following stimulus was applied for 60 minutes: 250 μA, 100 Hz, bipolar. This signal is similar to for SDF-1 stimulation (0.25 mA, a pulse duration of 40 pulses/s, a pulse width of 100 μs, and a frequency of 100 Hz for 1 or 4 h). SDF-1 stimulation can be related with stem cell migration and cell and tissue structure improvement, found in treated sheep.

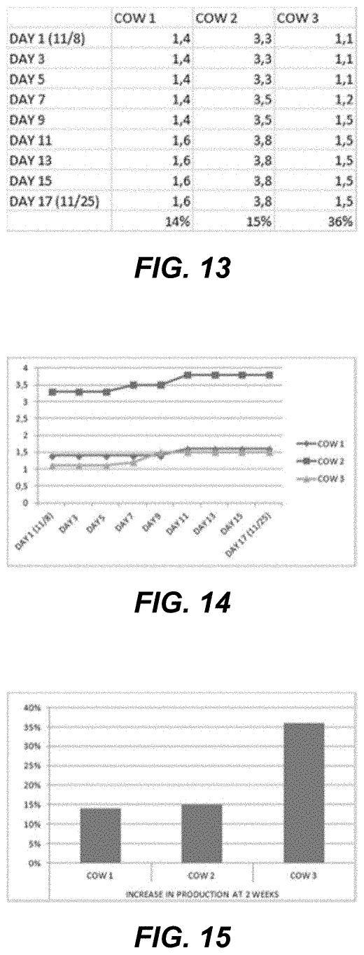

Milk production was monitored for the three cows. FIG. 13 depicts the raw data of milk production for the three cows in a table. FIG. 14 displays the results graphically. FIG. 15 shows the increase in production via a bar graph.

As can be seen, after about nine days, milk production from the treated udder began to increase. After two weeks, all three of the cows showed an increase of more than 10% in the milking volume from the treated udder. The animals did not display any adverse, general or local, effect(s).

All three cows showing similar improvements help greatly to verify the results.

### Example III

Biopsy samples and ultrasounds are taken from the sheep from EXAMPLE I and are analyzed. The process is found to be non-pathologic. Stem cell migration in addition to cell and tissue structural improvement has been found in samples from treated sheep. The veterinarian conducting the ultrasounds detected a real difference in favor of treated mammary glands.

### Example IV

The stimulation protocol from EXAMPLE I is administered to two cows (Aberdeen Angus). In two other cows, the stimulation period is reduced to 30 minutes, and the results are compared. One signal used was 250 μA; 100 Hz; bipolar; 60 minutes. The same signal was used for the other two cows, but the stimulation time is shortened. This signal is similar to SDF-1 (0.25 mA, a pulse duration of 40 pulses/s, a pulse width of 100 μs, and a frequency of 100 Hz for one or four hours).

The stimulation protocol from EXAMPLE I is used on two cows to stimulate milk producer cows in a dairy with an automatic milking system. Such cows are much more sensitive to stress. The cows that undergo automatic milking usually stay no more than ten minutes in the cowshed.

Shorter stimulation times are studied, particularly those administered immediately before milking takes place in order to avoid altering the farm routine.

Prolactin variation is measured using an appropriate ELISA kit.

### Example V

Conclusions from the veterinarian's report from EXAMPLE II.

Production:

Cow 1: went from 1.400 L to 1.700 (21.4% increase)

Cow 2 went from 3.4 liters to 3.9 liters (14.7% increase)

Cow 3 went from 1.1 liters to 1.4 liters (27.3% increase)

It is remarkable that the animal that had a minor production (cow 3) displayed a major stimulation in milk production. Also, is interesting that the animal that was producing more milk (cow 2) responded sooner (at day 4) to stimulation.

Preliminary conclusions:

1) There was a significant increase in milk production.

2) There was an ultrasound correlation.

3) There was a very evident increase in cellularity according to ultrasound evaluation.

4) There were no macroscopic changes in milk quality.

5) The treated udders showed consistency of productive tissue without evidence of scar tissue generation.

6) There were no animals that were refractory to the treatment.

### REFERENCES

(The contents of each of which is incorporated herein in its entirety by this reference.)
Prochazka et al., “Cocktail of Factors from Fat-derived Stem Cells Shows Promise for Critical Limb Ischemia,” http://www.sciencenewsline.com/news/2016012204520017. html (Jan. 21, 2016).
Salcedo et al., “Low current electrical stimulation up-regulates cytokine expression in the anal sphincter,” Int. J. Colorectal Dis., 2012 February; 27(2):221-5. doi: 10.1007/s00384-011-1324-3. Epub (October 2011).
“Electric Tumor Treatment Fields,” No. 0827 Policy, http://www.aetna.com/cpb/medical/data/800 899/0827.html (Nov. 18, 2016).
Hopkins Medicine “Overview of Pacemakers and Implantable Cardioverter Defibrillators (ICDs),” http://www.hopkinsmedicine.org/healthlibrary/conditions/cardiovascular diseases/overviewofpacemakersandimplantablecardioverterdefibrillators icds_85,P00234/.
Columbia “Implant Procedure Concepts—Pacemaker, ICD and CRT Overview,” http://www.columbia.edu/itc/hs/medical/hickey/docs/Pacemaker,%20ICD%20and%20CRT%20Overview%20022007.pdf
“FDA Approves Algovita Spinal Cord Stimulation System from Greatbatch,” http://www.odtmag.com/contents/view_breaking-news/2015-12-02/fda-approves-algovita-spinal-cord-stimulation-system-from-greatbatch (Dec. 2, 2015).
Mass Device “Greatbatch wins FDA PMA for Algovita SCS,” http://www.massdevice.com/greatbatch-wins-fda-pma-for-algovita-scs/ (Dec. 1, 2015).
Mosteiro et al., “Tissue damage and senescence provide critical signals for cellular reprogramming in vivo,” Science, 2016; 354 (6315): aaf4445 DOI: 10.1126/science. aaf4445.
Sutherland et al., “Prolonged electrical stimulation of the nipples evokes intermittent milk ejection in the anaesthetised lactating rat,” Exp Brain Res. 1987; 66(1):29-34.
Andersson & McCann “Drinking, antidiuresis and milk ejection from electrical stimulation within the hypothalamus of the goat,” Acta Physiol Scand. 1955 Dec. 31; 35(2):191-201; DOI: 10.1111/j.1748-1716.1955.tb01277.x.
Cross & Harris “Milk Ejection following Electrical Stimulation of the Pituitary Stalk in Rabbits,” Nature 166, 994-995 (9 Dec. 1950); doi:10.1038/166994b0.

1. A method of using a bioelectric stimulator that delivers at least one select electrical signal to a female subject's tissue, the method comprising:
delivering the select electrical signal(s) to or about the mammary glands or breasts of the subject so as to increase milk production in the subject.

2. The method according to claim 1, further comprising:
separately delivering to the subject a cocktail of agents comprising any combination of the following: stem cells, endothelial progenitor cells, selected exosomes, selected alkaloids, selected anti-inflammatory agents, nutrient hydrogel, organ specific matrix, selected growth factors, amniotic fluid, placenta fluid, cord blood, embryonic sourced growth factors and cells.

3. The method according to claim 1, wherein the electrical signal stimulates the production and/or release of at least one protein in the subject, the at least one protein selected from the group consisting of stromal cell-derived factor 1 (“SDF-1”), insulin-like growth factor 1 (“IGF-1”), vascular endothelial growth factor (“VEGF”), and any combination thereof.

4. The method according to claim 3, further comprising:
separately delivering to the subject stem cells and/or growth factors comprising any combination of SDF-1, IGF-1, and VEGF.

5. The method according to claim 1, wherein breast tissue generation or regeneration occurs in the subject.

6. The method according to claim 1, further comprising:
measuring the electrical activity of a series of bioelectric signals that stimulate milk production after suckling action on the breast nipple of the subject.

7. The method according to claim 6, further comprising:
mimicking the measured electrical activity to the subject this same series of bioelectric signals back to the subject for an extended period of time.

8. The method according to claim 7, further comprising:
amplifying this same series of bioelectric signals for application to the subject.

9. The method according to claim 1, wherein the at least one select electrical signal is 250 μA, 100 Hz, bipolar, applied for 60 minutes every other day for a month.

10. The method according to claim 1, wherein the at least one select electrical signal is 250 μA, 100 Hz, bipolar, applied for 30 minutes every other day for a month.

11. A method of increasing milk production in a suitable female subject, the method comprising:
delivering a selected electrical signal or signals to the subject so as to increase milk production in the subject,
wherein the selected electrical signal(s) comprise(s) a bioelectric signal of 250 μA, 100 Hz, bipolar, and
wherein milk production in the subject increases by at least 10% by volume after two weeks of delivering the selected electrical signal(s).

12. The method according to claim 11, wherein the selected electrical signal is 250 μA, 100 Hz, bipolar, applied for 60 minutes every other day for a month.

13. The method according to claim 11, wherein the selected electrical signal is 250 μA, 100 Hz, bipolar, applied for 30 minutes every other day for a month.

14.-15. (canceled)

16. The method according to claim 1, wherein the at least one select electrical signal is 250 μA, 100 Hz, bipolar.

17. The method according to claim 16, wherein the at least one select electrical signal is applied to the subject for at least two weeks.

18. The method according to claim 2, wherein the electrical signal stimulates the production and/or release of at least one protein in the subject, the at least one protein selected from the group consisting of stromal cell-derived factor 1 (“SDF-1”), insulin-like growth factor 1 (“IGF-1”), vascular endothelial growth factor (“VEGF”), and any combination thereof.

19. The method according to claim 2, wherein breast tissue generation or regeneration occurs in the subject.

20. The method according to claim 2, further comprising:
measuring the electrical activity of a series of bioelectric signals that stimulate milk production after suckling action on the breast nipple of the subject.

21. The method according to claim 2, wherein the at least one select electrical signal is 250 μA, 100 Hz, bipolar, applied for 60 minutes every other day for a month.

22. The method according to claim 2, wherein the at least one select electrical signal is 250 μA, 100 Hz, bipolar, applied for 30 minutes every other day for a month.
